(19) United States
(12) Patent Application Publication
(10) Pub. No.: US 2026/0273235 A1
(43) Pub. Date: Sep. 17, 2026

(54) NOVEL SELDINGER TECHNIQUE ACCESS WITH MICROVALVE ASSEMBLY

(71) Applicant: EVA INNOVATIONS, INC., Aventura, FL (US)

(72) Inventors: Daniel B. Stone, Aventura, FL (US); Jeffrey A. Panozzo, Naples, FL (US)

(73) Assignee: EVA INNOVATIONS, INC., Aventura, FL (US)

(21) Appl. No.: 19/662,628

(22) Filed: Apr. 29, 2026

Related U.S. Application Data
(63) Continuation-in-part of application No. 18/667,465, filed on May 17, 2024.
(60) Provisional application No. 63/865,802, filed on Aug. 18, 2025.
(60) Provisional application No. 63/797,237, filed on Apr. 30, 2025.
(60) Provisional application No. 63/515,273, filed on Jul. 24, 2023.

(51) Int. Cl. A61M 25/09; A61B 34/20; A61B 90/00; A61M 25/06; A61M 39/06; A61M 39/00

(52) U.S. Cl. CPC A61M 25/09041; A61B 34/20; A61B 90/37; A61M 25/0662; A61M 25/0693; A61M 39/06; A61B 2034/2046; A61B 2090/3762; A61B 2090/378; A61M 2039/0009; A61M 2039/0626; A61M 2205/0216; A61M 2205/586; A61M 2210/12

(57) ABSTRACT

Passive flashback devices enable one-handed guidewire insertion into a patient. A first device includes an elongate housing with a handle, a guidewire placement assembly with a wire feed channel, a passive guidewire feed surface, and a sheath brake, with or without an elastomeric microvalve assembly in the wire feed channel. A second device adds a guidewire carriage that slides along the housing between open and closed positions, with the microvalve assembly situated in a channel through a stanchion of the guidewire placement assembly. In both devices the guidewire moves freely and independently through the device without generating a vacuum. The microvalve assembly seals around the guidewire to prevent air embolism while allowing the operator to advance the guidewire with one hand. The operator's free hand simultaneously manipulates an ultrasound probe or other imaging device, improving speed and accuracy in interventional procedures including central line placement, nephrostomy, and vascular access.

### CROSS-REFERENCE TO RELATED APPLICATIONS

This application is a continuation-in-part of U.S. Patent Application Serial No. 18/667,465, filed May. 17, 2024, which claims the benefit of priority under 35 U.S.C. § 119(e) to U.S. Provisional Patent Application No. 63/515,273, filed Jul. 24, 2023. This application also claims the benefit of priority under 35 U.S.C. § 119(e) to U.S. Provisional Patent Application No. 63/797,237, filed Apr. 30, 2025 and U.S. Provisional Patent Application No. 63/865,802, filed Aug. 18, 2025. The entire disclosures of these applications are incorporated herein by reference.

### TECHNICAL FIELD

The present invention relates to devices for inserting a guidewire into a patient in connection with interventional procedures which often use ultrasound to image the position of a needle, guidewire or a more complex device in the patient. The present invention provides passive one-handed flashback devices for inserting a guidewire into the patient, as well as improved syringe-based devices with enhanced sealing mechanisms, and methods for treating medical conditions by medical practitioners utilizing the devices.

### BACKGROUND OF THE DISCLOSURE

WO 2018/089758 discloses a device for one-handed insertion of a guidewire with active flashback generated with vacuum assist from a syringe. Other references of interest include United States Patent No. 4,813,938 to Raulerson; United States Patent No. 6,485,428 to Enk Col 2, lines 45-57; United States Patent No. 6,217,558 to Zadini et al., Col. 12, lines 54-67; United States Patents Nos. 11,413,433 and 11,285,301 both to Vargas et al; and United States Patent Publication No. 2015/0314104 of Almansouri et al.

One passive flashback insertion device is shown in United States Patent No. 10,092,731 to Almansouri et al. It is noted in Col. 9, lines 3-8 the construction of the device only allows for inserting catheters shorter than the needle, which makes the device impractical and near impossible for procedures requiring a catheter longer than the needle. Many interventional procedures do require a longer catheter. A typical central line catheter has a length of 15-30 cm (6-12 inches) or so, too long for standard hypodermic needles, and which extended needle length would certainly cause patient distress during procedures where the patient remains conscious as well as difficulty threading/placing the catheter into the patient.

With respect to valve configurations generally, see United States Patent No. 8,262,623 to Nijland et al., as well as United States Patent Publication No. 2017/0002941 of Nijland et al. See also United States Patents Nos. 8,926,564 to King et al. and 11,839,735 to Bierman as well as United States Patent Application Publication No. 2018/0296804 to Bierman.

There exists a need in the art for improved devices adapted for one-handed operation that can be operated with the guidewire pre-installed and provide improved sealing to prevent air embolism while maintaining ease of guidewire advancement. Existing devices are also unnecessarily large and unwieldy, oftentimes exhibiting a lever effect which is difficult to control, particularly when the operator must simultaneously manipulate an imaging probe. A more compact design with the trigger positioned closer to the needle reduces the lever effect and gives the operator increased control during the procedure.

### SUMMARY OF THE INVENTION

The present invention addresses the need for improved devices adapted for one-handed guidewire insertion by providing passive flashback devices that avoid guidewire length limitations through the use of an untethered guidewire, and that use in some embodiments an elastomeric microvalve assembly. The inventive devices are more compact than prior art devices, typically about 6.5 inches or less in length as compared to 8 inches and 9 inches for prior art devices, and exhibit a significant reduction in the lever effect compared to prior art devices. The trigger is positioned closer to the needle than in prior art devices, giving the operator increased control and allowing one-handed operation while the other hand simultaneously manipulates an ultrasound probe or other imaging device.

In one embodiment, the present invention provides a device for introducing a guidewire into a patient configured for one-handed operation comprising: an elongate housing having (i) a handle at its rearward end, (ii) a guidewire placement assembly at its forward end with a wire feed channel communicating with a mounting projection for a hollow needle, said elongate housing further defining an upwardly open guidewire feed area; a guidewire passive feed surface disposed rearwardly of the guidewire placement assembly and vertically aligned with the upwardly open guidewire feed area; a guidewire sheath brake disposed rearwardly of the guidewire passive feed surface. In some embodiments, an elastomeric microvalve assembly is disposed in the wire feed channel. Preferably the passive feed surface is on a rotatably mounted wheel and the wire feed channel is conical or tubular. The device may be used in combination with a hollow needle affixed to the mounting projection and a guidewire inserted through the device such that the sheath engages the sheath brake and unsheathed guidewire extends over the guidewire passive feed surface and through the hollow needle, whereby the guidewire can be advanced into the patient with one hand.

In another embodiment, a passive flashback device for introducing a guidewire into a patient configured for one-handed operation comprises: an elongate housing having (i) a handle at its rearward end, (ii) a guidewire placement assembly at its forward end including a stanchion with a channel therethrough communicating with a hollow needle mounting projection which has a hollow needle mounted thereon communicating with the channel and (iii) said elongate housing further defining an upwardly open guidewire feed area; a guidewire carriage slidingly coupled to said elongate housing, the guidewire carriage having on its upper surface (i) a sheath brake, (ii) a wire feed channel and (iii) a passive guidewire feed surface, the guidewire carriage being positioned such that the wire feed channel and passive guidewire feed surface are vertically aligned with the upwardly open guidewire feed area of the housing, the guidewire carriage being movable between an open position where the wire feed channel is spaced away from the channel on the stanchion of the guidewire placement assembly and a closed position where the wire feed channel engages the channel on the stanchion; a sheathed guidewire engaged with the sheath brake such that the sheath of the guidewire cannot move forwardly with respect to the sheath brake, and the guidewire, which is freely movable within the sheath, extends forwardly with respect to the sheath brake, over the passive guidewire feed surface, through the wire feed channel, the channel in the stanchion and through the hollow needle; wherein the guidewire is untethered to the device and wherein the device does not include means for generating a vacuum. In some embodiments, an elastomeric microvalve assembly is disposed in the channel in the stanchion. Preferably the passive guidewire feed surface is on a freely rotating wheel and the wire feed channel is a conical channel. The guidewire carriage may have a trigger attached thereto, preferably a Y-shaped trigger, or alternatively a trigger assembly slidingly coupled to the guidewire carriage, configured to move the guidewire carriage forwardly and rearwardly. In preferred embodiments, the channel through the sheath brake, the passive guidewire feed surface, the wire feed channel, the channel through the stanchion, and the hollow needle are all substantially colinear.

In another embodiment, an assembly for receiving a sheathed guidewire in a passive flashback device for introducing a guidewire into a patient configured for one-handed operation comprises: an elongate housing having (i) a handle at its rearward end, (ii) a guidewire placement assembly at its forward end including a stanchion with a channel therethrough communicating with a hollow needle mounting projection which has a hollow needle mounted thereon communicating with the channel, and (iii) said elongate housing further defining an upwardly open guidewire feed area; and a guidewire carriage slidingly coupled to said elongate housing, the guidewire carriage having on its upper surface (i) a sheath brake, (ii) a wire feed channel and (iii) a passive guidewire feed surface, the guidewire carriage being positioned such that the wire feed channel and passive guidewire feed surface are vertically aligned with the upwardly open guidewire feed area of the housing, the guidewire carriage being movable between an open position where the wire feed channel is spaced away from the channel on the stanchion of the guidewire placement assembly and a closed position where the wire feed channel engages the channel on the stanchion of the guidewire placement assembly. In some embodiments, an elastomeric microvalve assembly is disposed in the channel through the stanchion. The guidewire carriage may have a trigger attached thereto or a trigger assembly slidingly coupled thereto configured to move the guidewire carriage forwardly and rearwardly.

In some embodiments, the elastomeric microvalve assembly includes at least one microvalve, preferably at least two microvalves, positioned to create a seal around the guidewire while still allowing the guidewire to advance freely through the device. The microvalve assembly may include a rearward dome valve having a slit seal and a back-up valve facing the needle with a cylindrical seal, both preferably made of ISO 10993 medical silicone or other suitable elastomeric compounds. The guidewire has a degree of interference with one or both seals of anywhere from about 15% to 80%, with from about 35% to about 55% being more preferred.

The present invention also provides improved medical procedures for the diagnosis and/or treatment of a medical condition comprising inserting a hollow needle into a patient, observing the needle enter the lumen or vessel utilizing an imaging modality while the needle is being advanced to confirm placement of the needle, optionally observing fluid or blood flashback to a hub of the needle, and subsequently advancing a guidewire through the needle into the patient while continuing to image the position of the guidewire in the patient with the imaging modality, the improvement comprising utilizing a passive flashback device of the invention to insert the guidewire. In some embodiments, the hollow needle may be inserted into a solid mass, such as a tumor. The imaging modality may be selected from the group consisting of ultrasound, fluoroscopy, X-ray, and computed tomography (CT) scanning,. The procedure may be used when the medical procedure is selected from the group consisting of central line placement, nephrostomy placement, uterine fibroid embolization, tumor ablation, embolization of the blood supply to a tumor, endovenous ablation for varicose veins, deep vein thrombosis treatment, IVC filter removal, and peripheral arterial disease treatment, as well as any other procedure involving the Seldinger technique or fluid draw.

Further details are provided in the annexed drawings and the text which follows.

### BRIEF DESCRIPTION OF THE DRAWINGS

The invention is described in detail below with reference to the drawings, wherein like numerals designate similar parts and wherein:

FIG. 1 is a view in perspective of a syringe-based insertion device according to an embodiment of the present invention with a guidewire installed, without a needle;

FIG. 2 is a view in perspective of the device of FIG. 1 with the guidewire extended;

FIG. 3 is a view in perspective of the device of FIG. 1, without the syringe installed;

FIG. 4 is a view in perspective and longitudinal section of the device of FIG. 1;

FIG. 5A is a side view of the device of FIG. 1, without a syringe, wherein the carriage is in a forwardmost position;

FIG. 5B is a side view of the device of FIG. 1, without a syringe, wherein the carriage is in a fully retracted position;

FIG. 6 is a sectional view of the syringe of FIG. 2 with the guidewire installed through the syringe;

FIG. 7 is a sectional view of the valve assembly components in the plunger of FIG. 1 in a partially assembled and uncompressed state;

FIG. 8 is a view in elevation of the plunger in the device of FIG. 1, wherein the valve assembly is installed in a compressed state;

FIG. 9 is a perspective view of a passive flashback device according to another embodiment of the present invention;

FIG. 10 is a left side view in elevation of the passive flashback device of FIG. 9;

FIG. 11 is a right side view in elevation of the passive flashback device of FIG. 9;

FIG. 12 is a front view in elevation of the passive flashback device of FIG. 9;

FIG. 13 is a rear view in elevation of the passive flashback device of FIG. 9;

FIG. 14 is a top plan view in elevation of the passive flashback device of FIG. 9;

FIG. 15 is a bottom plan view in elevation of the passive flashback device of FIG. 9;

FIG. 16 is a view in elevation and cross section of the passive flashback device of FIG. 9 along a centerline of the device;

FIG. 17 is a partial view in elevation and cross section of the passive flashback device of FIG. 9 along a centerline of the device with an elastomeric microvalve assembly installed about a guidewire;

FIG. 18 is a schematic enlarged view of the microvalve assembly installed in FIGS. 17 and 36 about a guidewire;

FIG. 19 is an exploded perspective view of an alternate assembly of a passive flashback device of the present invention;

FIG. 20 is an exploded left side view in elevation of the passive flashback device of FIG. 19;

FIG. 21 is a perspective view of the flashback device of FIG. 19 wherein the elongated housing and a guidewire carriage of the device are slidingly coupled to each other, and wherein the assembly is in a closed position;

FIG. 22 is a perspective view of the flashback device of FIG. 19 wherein the elongated housing and a guidewire carriage of the device are slidingly coupled to each other, and wherein the assembly is in an open position;

FIG. 23 is a left side view in elevation of the flashback device of FIG. 19 wherein the elongated housing and a guidewire carriage of the device are slidingly coupled to each other, and wherein the assembly is in an open position;

FIG. 24 is a left side view in elevation of the flashback device of FIG. 19 wherein the elongated housing and a guidewire carriage of the device are slidingly coupled to each other, and wherein the assembly is in a closed position;

FIG. 25 is a front view in elevation of the flashback device of FIG. 19 wherein the elongated housing and a guidewire carriage of the device are slidingly coupled to each other;

FIG. 26 is a rear view in elevation of the flashback device of FIG. 19 wherein the elongated housing and a guidewire carriage of the device are slidingly coupled to each other;

FIG. 27 is a top plan view of the flashback device of FIG. 19 wherein the elongated housing and a guidewire carriage of the device are slidingly coupled to each other in an open position;

FIG. 28 is a bottom plan view of the flashback device of FIG. 19 wherein the elongated housing and a guidewire carriage of the device are slidingly coupled to each other in a slightly open position;

FIG. 29 is a top plan view of the flashback device of FIG. 19 wherein the elongated housing and a guidewire carriage of the device are slidingly coupled to each other in a closed position;

FIG. 30 is a bottom plan view of the flashback device of FIG. 19 wherein the elongated housing and a guidewire carriage of the device are slidingly coupled to each other in a closed position;

FIG. 31 is a left side view in elevation and cross section of the passive flashback device of FIG. 19 along a centerline of the device of the flashback device of FIG. 19, wherein the elongated housing and a guidewire carriage of the device are slidingly coupled to each other, and wherein the assembly is in an open position;

FIG. 32 is a left side view in elevation and cross section of the passive flashback device of FIG. 19 along a centerline of the device of the flashback device of FIG. 19, wherein the elongated housing and a guidewire carriage of the device are slidingly coupled to each other, and wherein the assembly is in a closed position;

FIG. 33 is a left side view in elevation of the passive flashback device of FIG. 19 in a closed position with a guidewire and needle installed on the device;

FIG. 34 is an illustration the passive flashback device of FIG. 33 in the closed position grasped by a user, wherein it is seen that the device is operated with one hand, with digits of the user holding the handle, one digit of the same hand operating the trigger and another digit of the same hand adjacent a passive guidewire feed surface of the device which said other digit can be used to advance the guidewire, it is appreciated from FIG. 34 and the discussion herein that the housing, guidewire carriage, the guidewire and sheath brake, the wire feed channel, and needle are all positioned, configured and adapted for one-handed placement of a needle and guidewire in a patient;

FIG. 35 is a schematic diagram illustrating the importance of conical channels in avoiding unwanted blockages of the guidewire in the device due to the J bend on the end of a guidewire;

FIG. 36 is a schematic diagram illustrating a microvalve assembly installed on the device of FIGS. 19, 35;

FIG. 37 is a perspective view of another alternate assembly of a passive flashback device of the present invention;

FIG. 38 is a left side view in elevation and cross section of the passive flashback device of FIG. 37 along a centerline of the device of the flashback device of FIG. 37, wherein the elongated housing and a guidewire carriage of the device are slidingly coupled to each other, and wherein the assembly is in a closed position;

FIG. 39 is a left side view in elevation of the flashback device of FIG. 37 wherein the elongated housing and a guidewire carriage of the device are slidingly coupled to each other, and wherein the assembly is in a closed position;

FIG. 40 is a right side view in elevation of the flashback device of FIG. 37 wherein the elongated housing and a guidewire carriage of the device are slidingly coupled to each other, and wherein the assembly is in a closed position;

FIG. 41 is a front view in elevation of the flashback device of FIG. 37 wherein the elongated housing and a guidewire carriage of the device are slidingly coupled to each other;

FIG. 42 is a rear view in elevation of the flashback device of FIG. 37 wherein the elongated housing and a guidewire carriage of the device are slidingly coupled to each other;

FIG. 43 is a top plan view of the flashback device of FIG. 37 wherein the elongated housing and a guidewire carriage of the device are slidingly coupled to each other in a closed position;

FIG. 44 is a bottom plan view of the flashback device of FIG. 37 wherein the elongated housing and a guidewire carriage of the device are slidingly coupled to each other in a closed position;

FIG. 45 is a schematic diagram of a needle and guidewire placement procedure using a device of the invention and an ultrasound probe; and

FIG. 46 is a schematic diagram of inserting a central line (catheter) into a patient over a guidewire which has been placed in a patient.

### DETAILED DESCRIPTION

The present invention is described in detail below in connection with the annexed drawings. Terminology has its ordinary meaning; mm means millimeters, cc or ml means cubic centimeters and so forth, while more specific meaning is provided to certain terms and phrases, as appearing below.

When we refer to a conical channel, "conical" means that the channel has a conical portion or is conical over the length of the channel; preferably tapering to engage another channel and sized so as to prevent a guidewire with a J bend from relaxing in the interior of a channel and jamming the device. A conical channel may thus have tubular portions.

"Flashback" refers to blood or other bodily fluid flow out of the body, through a hollow needle inserted into the patient, to a needle hub attached to the hollow needle.

"Degree of interference" and like technology refers to the difference in guidewire diameter to seal diameter divided by seal diameter times 100%, or

((guidewire diameter - seal diameter) ÷ seal diameter) X 100%

"Air leak value" is expressed in percent as (air leak volume / syringe volume) X 100%. Air leak volume is determined by drawing a full syringe volume from a reservoir of deionized water at 25°C while disposing the syringe at approximately 45° from vertical with the guidewire installed in the syringe throughout the draw process. Thus, a 3cc syringe which exhibits 3/5cc of air leakage with the guidewire installed has an air leak value of (0.6/3) X 100% or 20%.

A "passive flashback device" refers to a device which does not have a vacuum source connected to or communicating with a hollow needle, rather flashback occurs spontaneously when the needle is placed in the patient, for example in the lumen of a blood vessel.

Microvalves are small components within the realm of fluidics and microfluidics designed to regulate gases or fluids. Their outer dimensions typically measure less than 10 mm, but may be somewhat larger depending upon a particular construction; generally microvalves are available with outer dimensions of up to about 40mm. A microvalve assembly includes at least 1 microvalve and most preferably at least 2 microvalves.

A "passive guidewire feed surface" refers to a surface used to guide a guidewire through a device without imparting forward or backward motion to the guidewire, such as a surface on a freely rotating guidewheel, or simply any surface that guides the wire without inducing or halting motion of the guidewire. In the case of a guidewheel, motion of the wire is in response to engagement with a digit of a user of the device who moves the wire forwardly or rearwardly with that digit pressed against the guidewire and guidewheel. A raised fixed surface is also contemplated, such that the guidewire is moved forwardly and rearwardly by a digit of an operator pressing the wire in a forward or rearward direction over the passive guidewire feed surface.

"Tubular" when referring to a channel means cylindrical in shape, unless otherwise specified.

Interventional medical procedures are used to treat a variety of conditions, including cancer and circulatory dysfunction. Interventional medical procedures use an ultrasound probe (or other imaging modality) to image the position of a needle, guidewire or a more complex device in the patient. The passive flashback to the needle hub or the posterior aspect of the guidewire assembly of the present invention also confirms needle placement.

The inventive devices can be manipulated with one hand during needle placement and advancing a guidewire, so the operator can use their other hand to utilize an imaging probe.

Guidewires are interventional procedure wires used to guide access, maneuver, or work through structures. There are many different types of wires that are used in medical procedures, but they aim to serve a few key roles:

Access wires: wires used temporarily to gain access into vessels/structures, often times quickly exchanged for other types of wires.

Maneuvering wires: used to gain entry into difficult to access vessels or structures.

Rail wires: these are more secure wires that are used as support for conducting interventions (advancing devices etc.).

Interventional medical procedures include:

A central line (central venous catheter) is a long, flexible tube. The tube is typically inserted into a vessel in the neck, chest, arm or groin. Common types of central lines include a peripherally inserted central catheter (PICC line), which is typically placed in the upper arm or near the elbow. Central lines may be coupled to an implanted port. This type of port is surgically placed into the chest and connected to a catheter that administers medication, draws blood and meets other needs, all occurring under the skin. This type of central line is used for patients needing treatment that will last for months. Placement of the central line is over a guidewire as discussed herein.

A central line provides access to a person's blood supply, which allows the patient to receive medications, fluids or additional blood, and allows practitioners to measure or draw blood. Central lines have many uses, including: receiving medicines that would damage smaller vessels or veins in the body, such as chemotherapy drugs; receiving high-pressure injections (through ports) needed for patients who require chronic red blood cell exchange transfusions, and administering contrast agents needed for computerized tomography (CT) or MRI scans; for frequent injections, use of IV medicines and blood draws during the course of treatment, resulting in fewer needle sticks (this can be particularly helpful for pediatric patients); and administering medications such as antibiotics more quickly during emergencies.

The devices of the invention may also be used for IV catheter placement, including over the needle catheter placement if so desired.

The devices of the present invention are used to insert guidewires into patients in connection with the following interventional procedures, whether or not it is necessary to observe flashback.

The devices of the present invention may be used for various interventional procedures including: nephrostomy placement for draining urine when ureters are blocked; uterine fibroid embolization (UFE) to treat fibroid tumors; interventional oncology procedures including tumor ablation, port insertion, and embolization; venous disease treatments including endovenous ablation for varicose veins, deep vein thrombosis (DVT) treatment via IVC filters or thrombolysis, IVC filter removal, and peripheral arterial disease (PAD) treatment via angioplasty, stent placement, or thrombolysis, as well as any other procedure involving a guidewire, such as retrograde intubation, chest tube placement, and blood draw.

In connection with procedures where flashback observation is needed to confirm needle placement, a passive flashback device is sufficient to confirm needle placement into the desired target, along with the imaging provided by ultrasound or equivalent radiology. The passive feedback devices of the invention eliminate the need for a syringe which is somewhat difficult to operate, requiring more advanced skill. One handed operation of the devices allows the operator to use their other hand to manipulate an ultrasound probe, as well as observe placement of the guidewire in real time, so that assistance from another person is not required. The one-handed devices of the invention also greatly reduce procedure time, alleviate patient discomfort and allow the medical provider to perform many more procedures in a day.

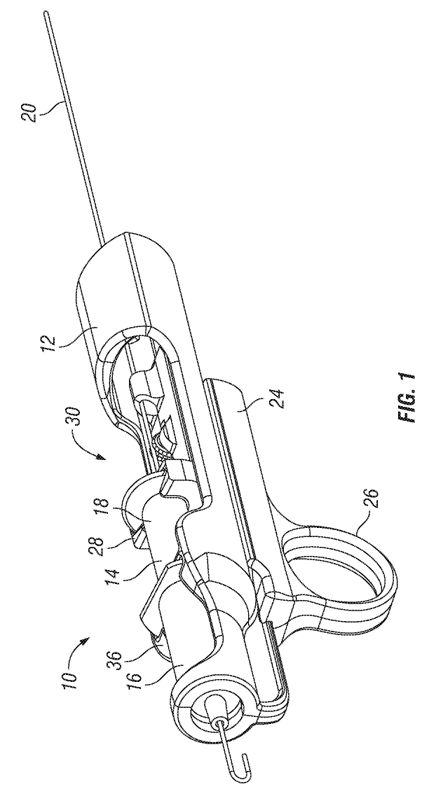

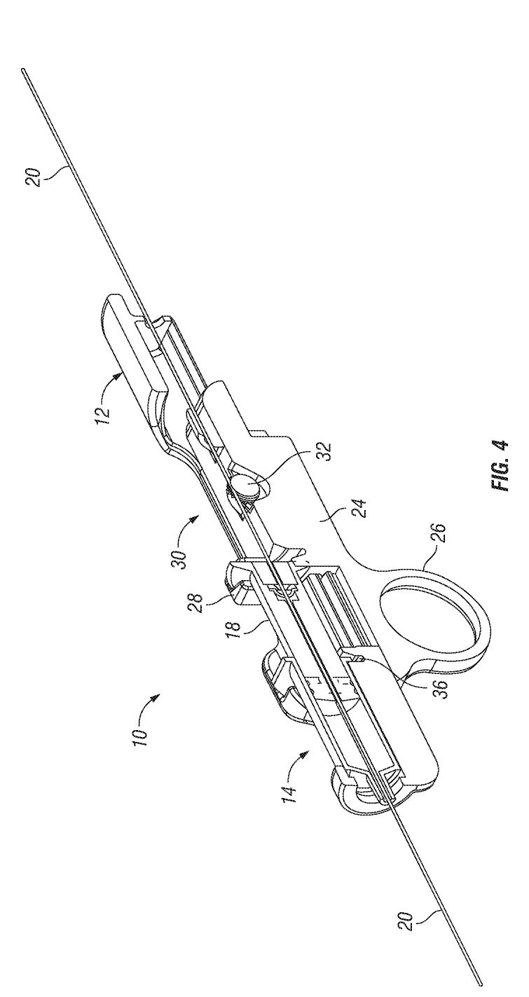

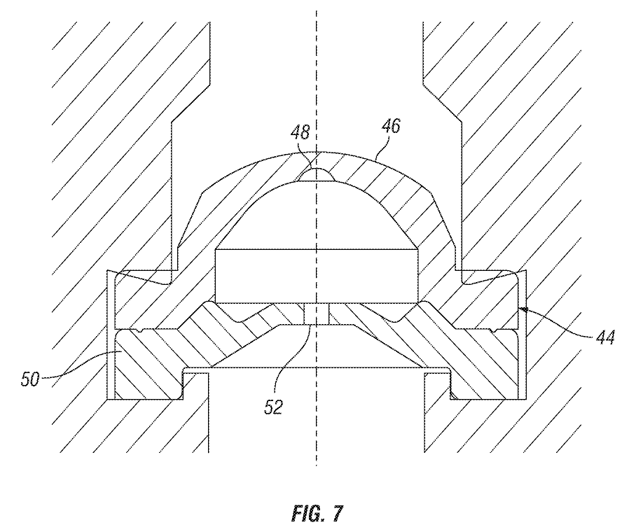

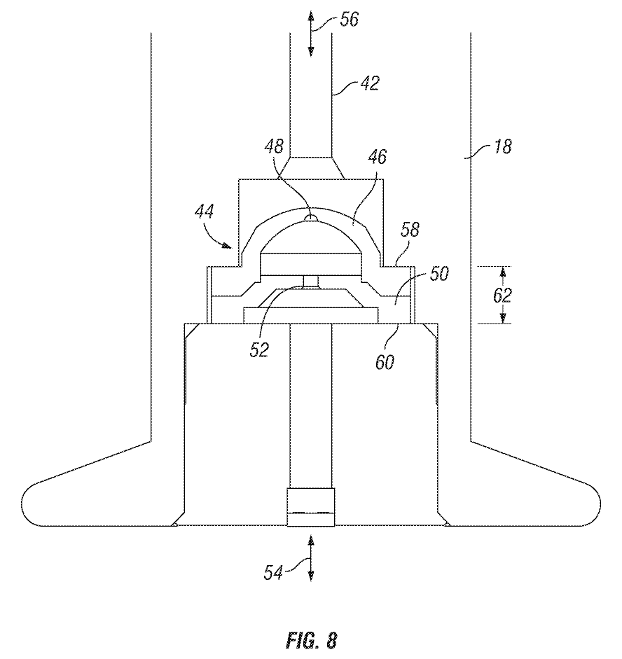

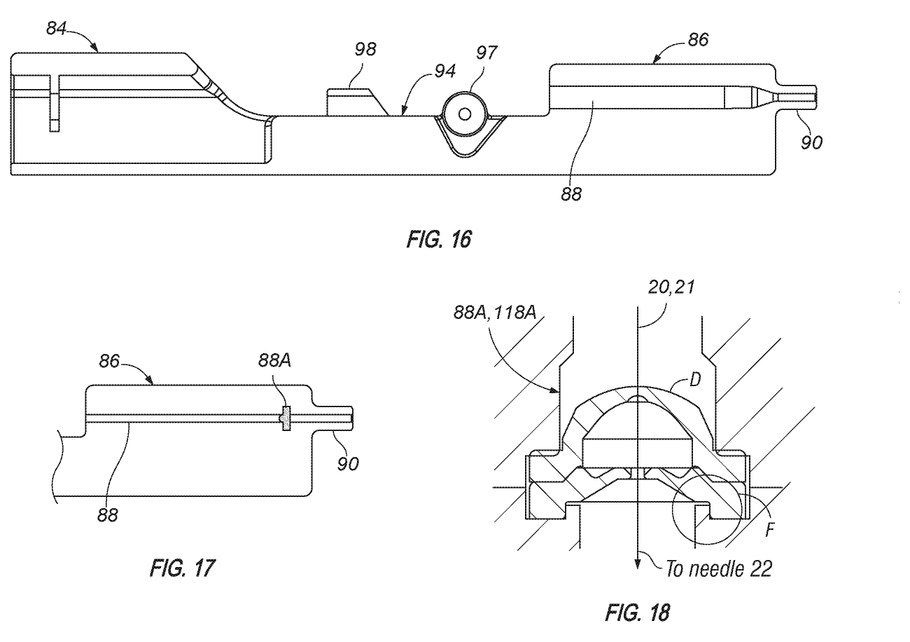

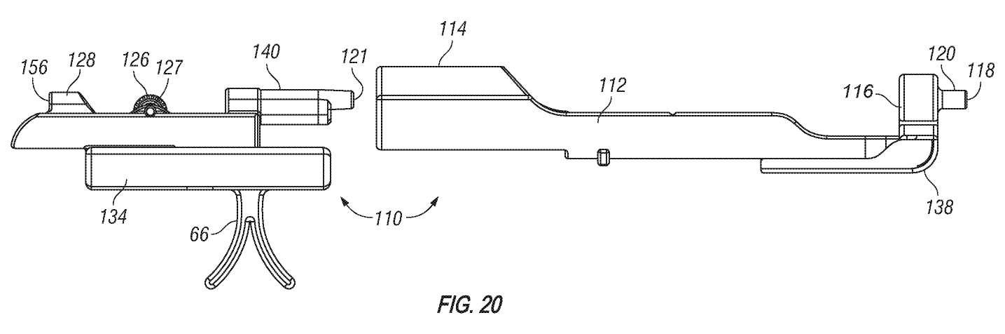

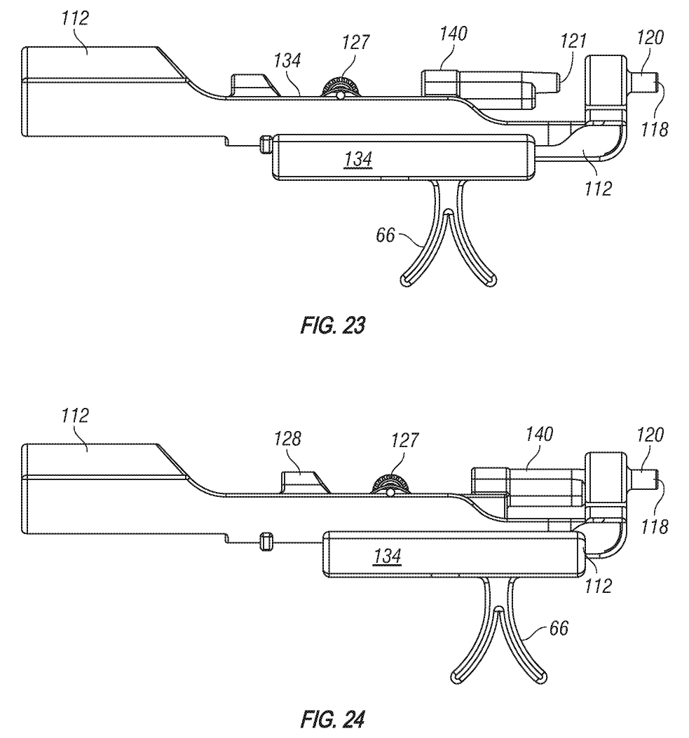

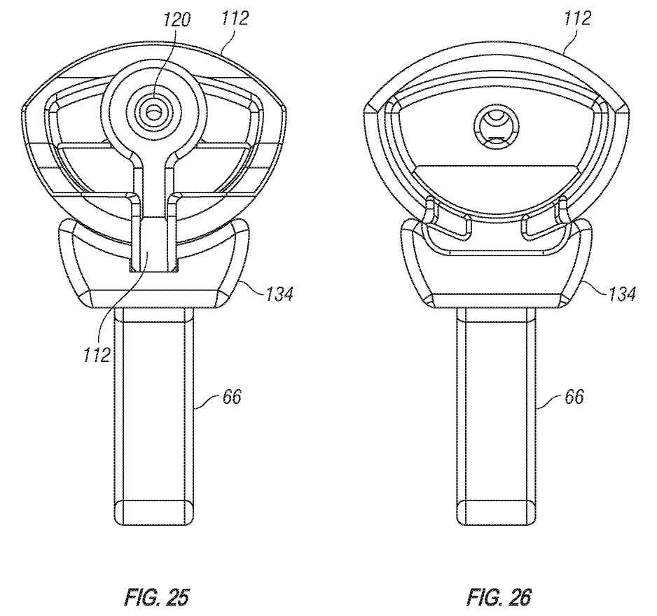

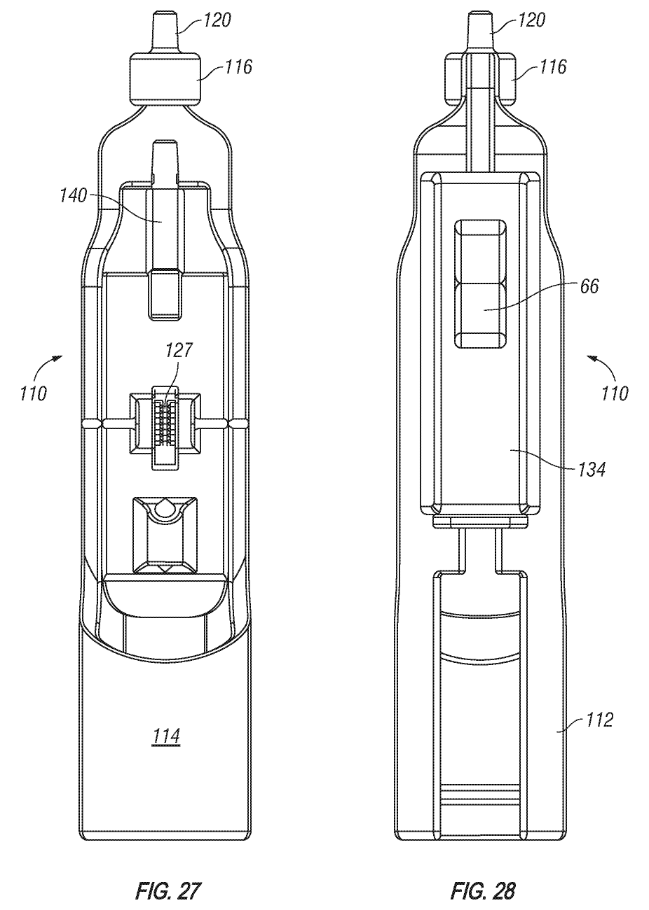

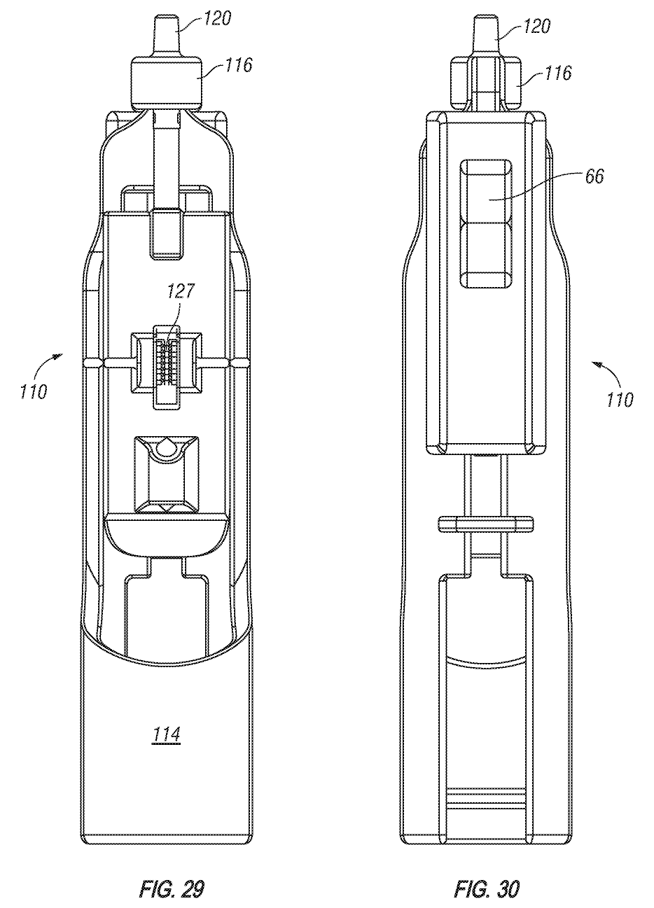

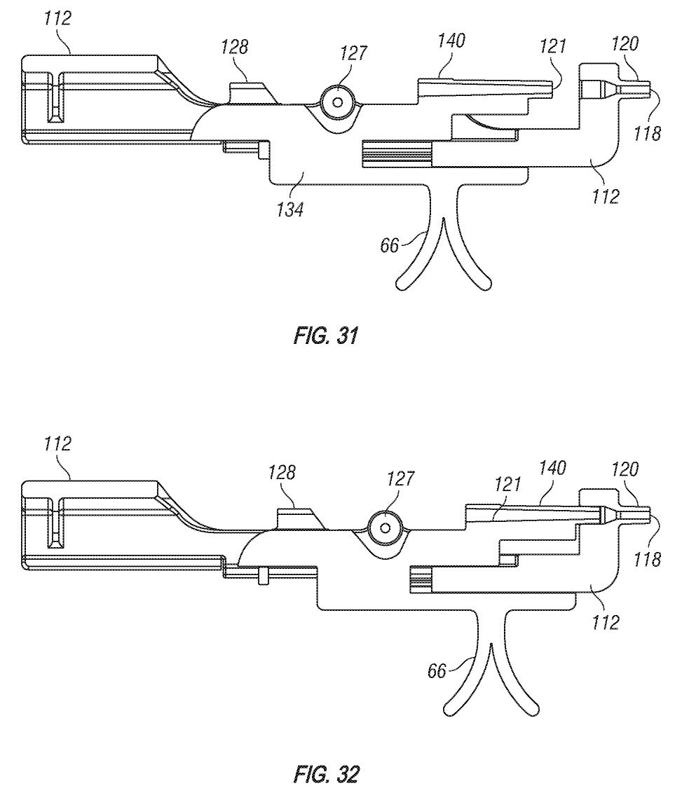

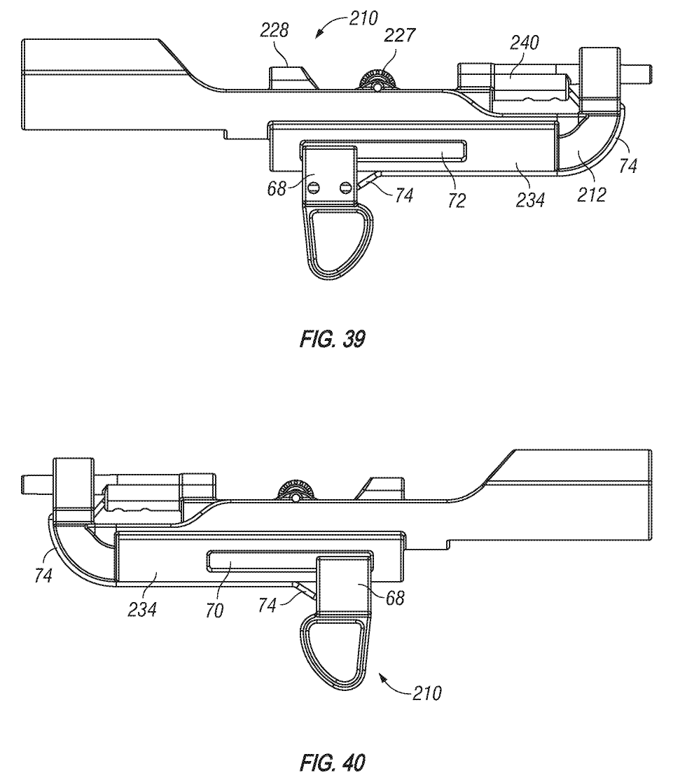

Turning to FIGS. 1 through 46, the invention is described below in connection with multiple embodiments. Referring first to FIGS. 1 through 8, there is illustrated a device 10 for the one-handed insertion of a guidewire 20 including a mounting/gripping member 12 having disposed thereon a syringe 14 with a barrel 16 and a plunger 18. Plunger 18 has further features, discussed below.

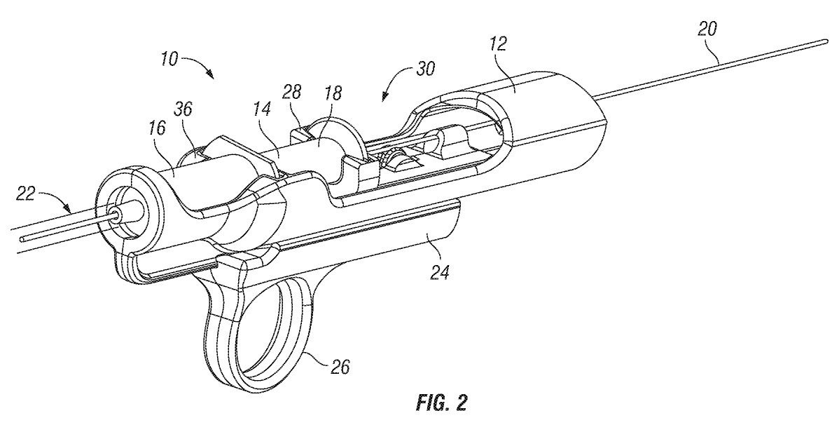

FIG. 1 shows device 10 with the guidewire 20 installed with "J" bend in the guidewire extending through the device, while FIG. 2 shows device 10 with guidewire 20 extending through device 10 and a hollow needle indicated schematically at 22.

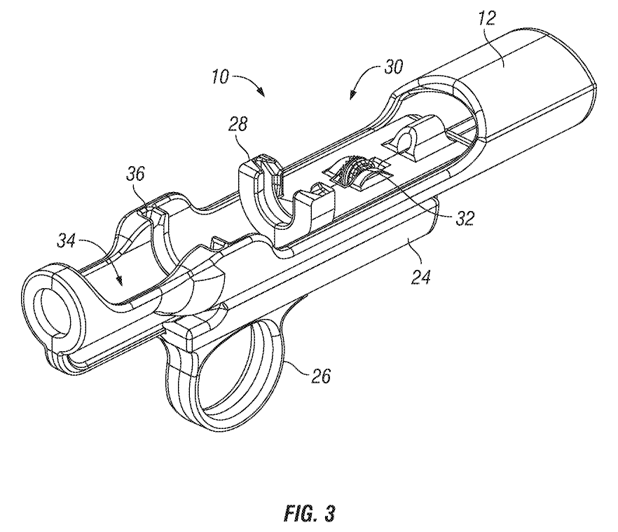

The various features of device 10 are further illustrated in FIGS. 3, 4 and 5.

Device 10 includes a carriage 24 slidably engaged to mounting/gripping member 12 having a trigger 26 and a flange trap 28 for a flange in the plunger, as well as a guidewire feed 30, including a guidewheel feed 32 (see FIG. 3).

Mounting/gripping member 12 includes a mounting area 34 for receiving the syringe including a barrel flange trap 36 for receiving the barrel in place on mounting/gripping member 12. Alternatively, barrel 16 may be integral with mounting/gripping member 12.

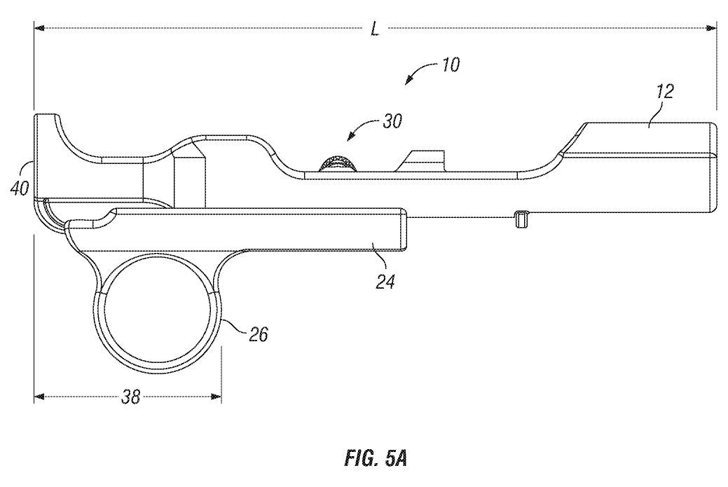

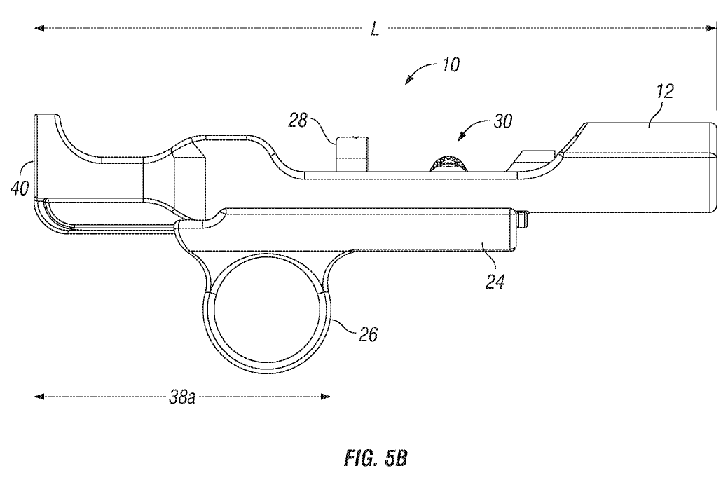

Gripping member 12 has a length, L, of 7" or less, typically from about 3" to about 7". FIG. 5A shows carriage 24 in a forwardmost position where a distance 38 of trigger 26 is at most about 2.25"; suitably, from about 0" to about 4.0". Distance 38 is measured from an end 40 of the mounting/gripping member 12 to the innermost projection of trigger 26 in its forwardmost position as indicated on FIG. 5A. Note that the carriage is considerably shorter than the mounting/gripping member.

In FIG. 5B carriage 24 is in a fully retracted position, wherein distance 38a is measured from end 40 of the mounting/gripping member 12 to a rearwardmost projection of trigger 26 in its fully retracted position. Distance 38a is anywhere from about 0.25" to about 5".

As alluded to earlier, a raison d'être of the present invention is to allow clinicians to practice a Seldinger technique using only one hand for both needle placement as well as for manipulation of the guidewire while using the other hand to position a visualization device, such as an ultrasound, thus enabling the clinician to ensure that the ultrasound is placed properly for optimum viewing of the needle/guidewire as it enters the lumen. In many cases, where a central line is needed, time will absolutely be of the essence and the ability of the clinician to insert the line with minimal delay is of the utmost importance.

In the Seldinger technique, a syringe having a beveled needle is inserted into the body with the point of the bevel engaging the skin first. Where body conformation allows, it is usually preferable that the needle of the syringe make an acute angle with the surface of the skin. The fixtures of the present invention enable the clinician to apply negative pressure by pulling back slightly on the plunger of the syringe with one hand whereas in the conventional technique, the use of 2 hands can be somewhat awkward or clumsy. The awkwardness can lead to incorrect placement of the guidewire. In many cases, the clinician will be able to manipulate the plunger using a single digit, perhaps the index finger, on the hand holding the fixture.

Once the guidewire is passing through the bore of the needle, the clinician will have increased ability to perceive the resistance being encountered by the guidewire (tactile perception). The clinician will also be able to use ultrasound continuously and more expeditiously, in contrast to prior methods in which the clinician would not be able to observe the guidewire enter the vessel without an assistant. The device of the present subject matter enables the clinician to perceive subtle variations in the degree of resistance, indicative of where the guidewire is passing. This can be extremely significant as it avoids the contingencies in which the clinician might be tempted to withdraw the guidewire slightly to re-orient it in the lumen of the vessel. In many cases, it is of course conventional for the guidewire to have a "J" shaped tip to guard against inadvertent perforation of a vessel wall.

After the guidewire is properly positioned, with the fixture of the present invention, the clinician moves the fixture rearwardly and only is required to begin using his other hand to restrain the guidewire as the bevel of the needle exits the body since the grip of surrounding tissue will be sufficient to hold the guidewire in place while the fixture is being withdrawn.

Once the guidewire has been properly positioned, and the means of inserting it have been cleared away, the clinician using the fixture of the present invention will proceed generally in accordance with the conventional procedure.

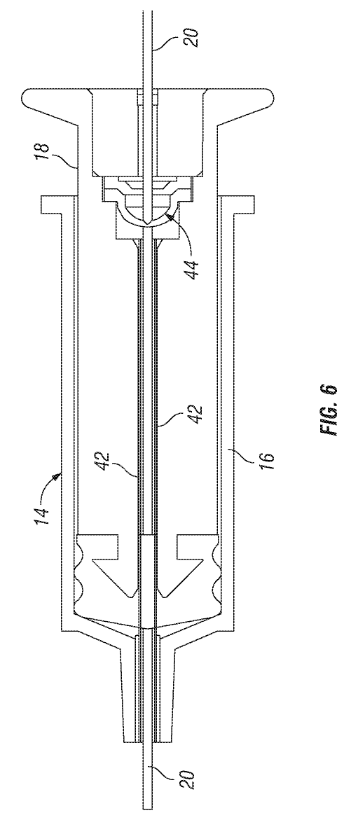

The present invention further facilitates practicing a one-handed Seldinger technique in that the device simplifies operation considerably due to its compact design and because the guidewire is pre-installed in the device, which in some embodiments requires improved sealing around the guidewire to prevent air and other fluid leakage as discussed below in connection with FIGS. 6, 7 and 8.

Syringe 14 has a perforate conduit 42 for receiving guidewire 20 secured within barrel 16 which slidably accommodates plunger 18.

A valve assembly 44 is disposed in the plunger. Assembly 44 includes a forward dome valve 46 having a slit seal 48, similar to a duck-bill valve, and a back-up valve 50 with a cylindrical seal 52. Both valves are most preferably made of ISO 10993 medical silicone.

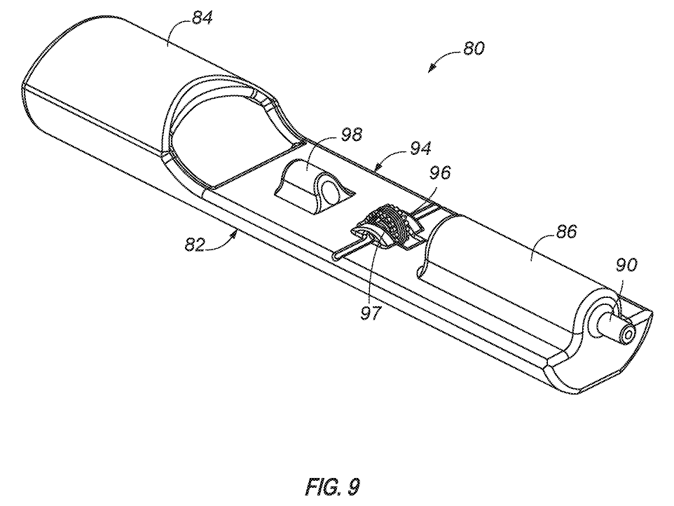

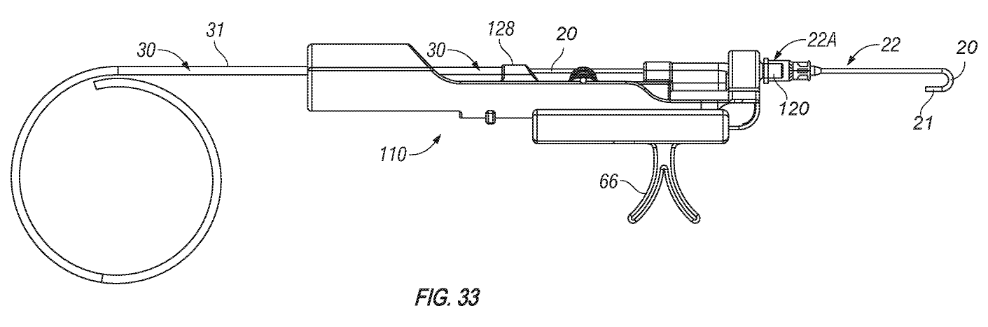

Referring to FIGS. 9 through 17, there is illustrated a device 80 for introducing a guidewire into a patient configured for one-handed operation consisting of: an elongate housing 82 having (i) a handle 84 at its rearward end, (ii) a guidewire placement assembly 86 at its forward end with a wire feed channel 88 communicating with a mounting projection 90 for a hollow needle 22 (see FIG. 33), said elongate housing further defining an upwardly open guidewire feed area 94. A guidewire passive feed surface 96 is disposed rearwardly of guidewire placement assembly 86 and vertically aligned with upwardly open guidewire feed area 94. Elongate housing 82 further provides a guidewire sheath brake 98 disposed rearwardly of guidewire passive feed surface 96.

Preferably, device 80 has passive feed surface 96 on a rotatably mounted wheel 97 and wire feed channel 88 through guidewire placement assembly 86 is conical or tubular.

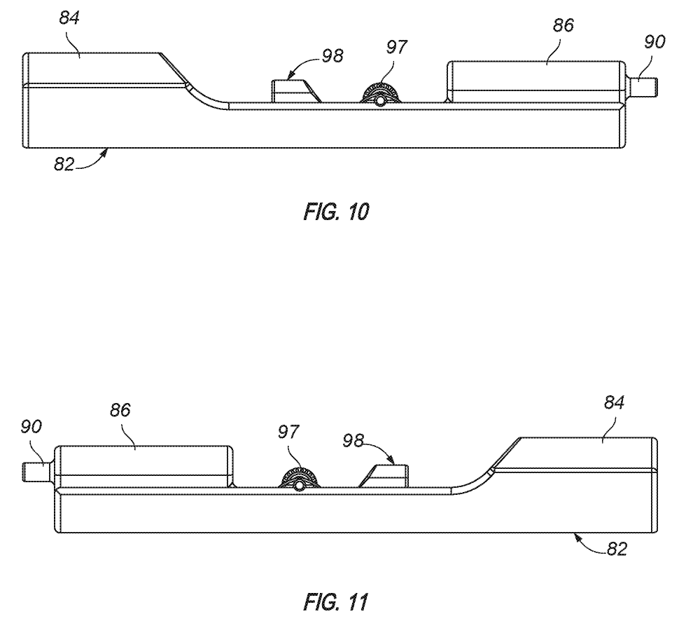

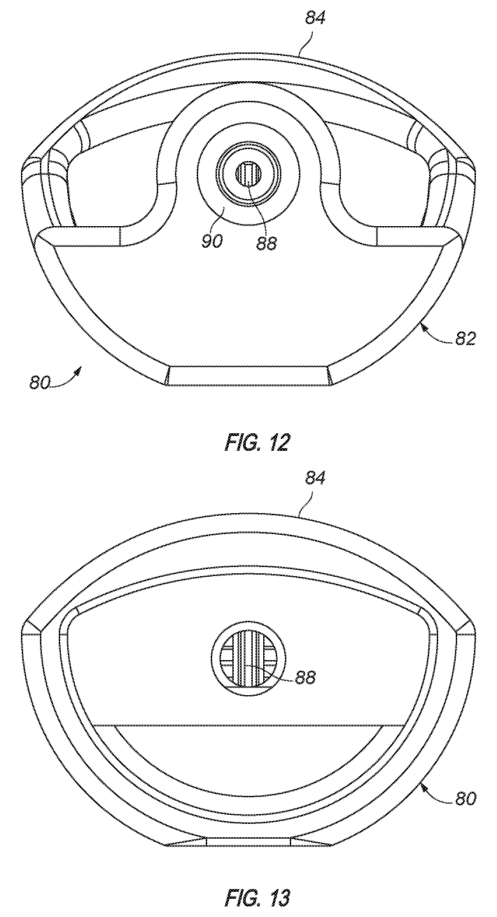

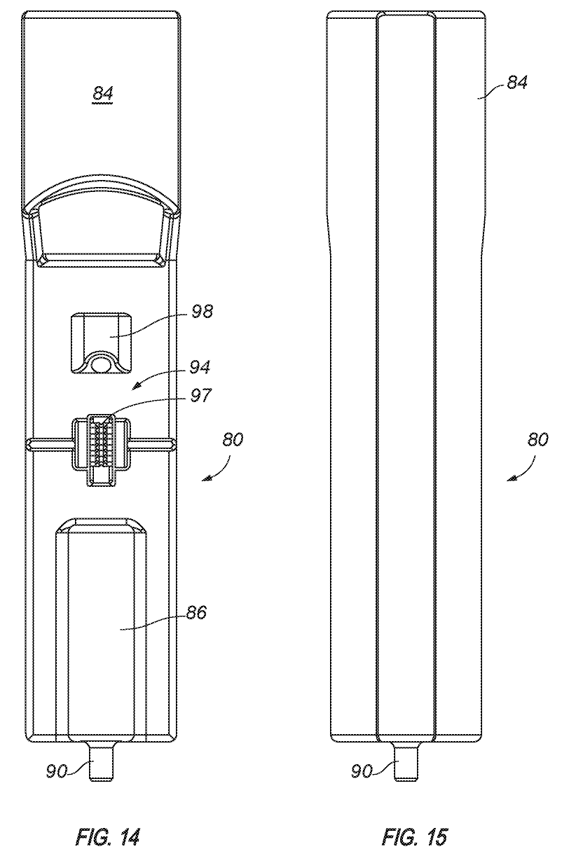

FIGS. 10 and 11 are left and right side views in elevation of device 80 of FIG. 9, respectively. FIG. 12 is a front view in elevation and FIG. 13 is a rear view in elevation of device 80. FIGS. 14 and 15 are top and bottom plan views of device 80, respectively. These views further illustrate the overall form of elongate housing 82, handle 84, guidewire placement assembly 86, passive guidewire feed surface 96 (see FIG. 9) on rotatably mounted wheel 97, and guidewire sheath brake 98.

Device 80 is used in combination with hollow needle 22 affixed to mounting projection 90 of guidewire placement assembly 86 and in further combination with a sheathed guidewire 30 inserted through the device, such that the sheath engages sheath brake 98, and guidewire 20 extends over the guidewire passive feed surface and through the hollow needle, whereby the guidewire can be advanced into the patient with one hand. See FIG. 33 and note the discussion relating thereto. Preferably guidewire 20 is untethered to other components of the combination.

A cylindrical wire feed channel 88 in the device which has a similar diameter to the needle would, most likely, negate the need for a valve, as the snugly situated guidewire in the feed channel 88 should prevent an air embolus. Nevertheless, vascular insertion devices sometimes include a valve or valve assembly to reduce or eliminate air leakage around the guidewire as is seen, for example, in United States Patents Nos.: 8,926,564; 11,839,735 as well as United States Patent Application Publication No. 20180296804.

FIG. 17 illustrates a microvalve assembly 88A installed in wire feed channel 88 of device 80. Further details of the microvalve assembly appear in FIG. 18 and the discussion relating thereto. In use, the guidewire is threaded through microvalve assembly 88A, which has at least one elastic seal in an interference fit with the guidewire. Preferably, at least one elastic seal in an interference fit with the guidewire has a degree of interference with the guidewire from about 15% to about 80%, with from about 35% to about 55% being more preferred. A guidewire with a diameter of 0.80mm through a microvalve with a cylindrical seal having a diameter of 0.55mm has a degree of interference of about 45% with respect to the seal. More preferably, at least one elastic seal in an interference fit with the guidewire has a degree of interference with the guidewire of at least about 25%. The at least one elastic seal in an interference fit with the guidewire is made of a material selected from the group consisting of a rubber, a thermoplastic elastomer, an ISO 10993 medical grade silicone, and any combination thereof. Besides the degree of interference of the guidewire with the elastic seal, it is also important not to distort the seals during assembly of the devices. To this end, the retaining surfaces do not compress the valves more than about 7.5–15% from their original dimensions, regardless of assembly method, whether threaded and screwed together or snap-fit with appropriate features on the device components. To this end, the retaining surfaces are of a predetermined axial distance with respect to one another of no less than about 85%–92.5% of the thickness of the peripheries of the valve assembly components prior to compression.

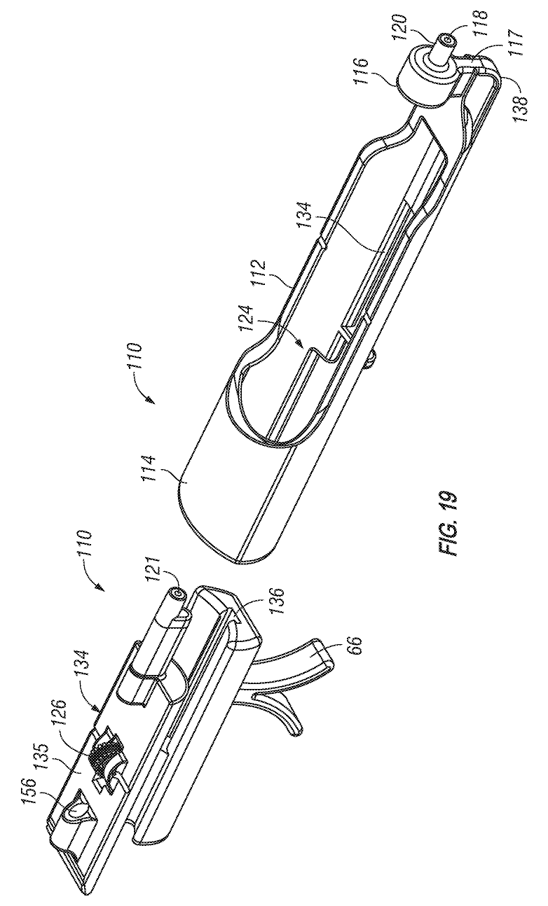

Referring to FIGS. 19 through 36, there is illustrated another passive flashback device 110 for introducing a guidewire into a patient configured for one-handed operation including (a) an elongate housing 112 having (i) a handle 114 at its rearward end, (ii) a guidewire placement assembly 116 at its forward end including a stanchion 117 with a channel 118 therethrough communicating with a hollow needle mounting projection 120 which has a hollow needle 22 mounted thereon communicating with channel 118 and (iii) said elongate housing further defining an upwardly open guidewire feed area 124; and (b) a guidewire carriage 134 is slidingly coupled to said elongate housing by way of a plurality of mounting grooves 136 and a plurality of ridges 138 on the carriage and the housing.

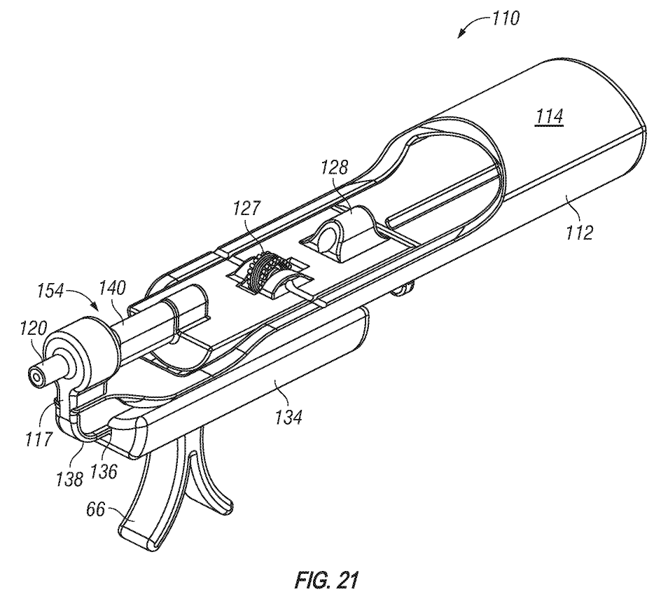

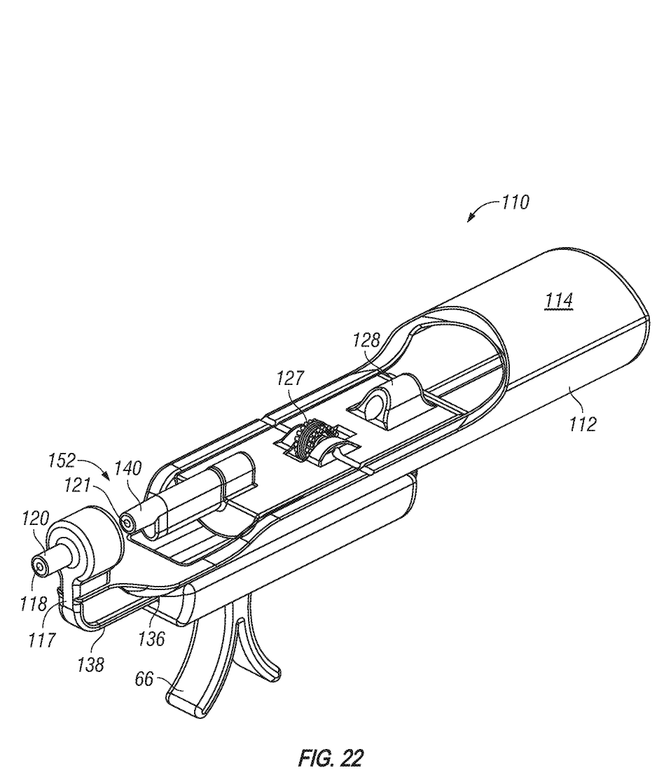

Guidewire carriage 134 has on its upper surface 135 (i) a sheath brake 128, (ii) a wire feed channel 121 extending through a feed tube 140 and (iii) a passive guidewire feed surface 126, the guidewire carriage being positioned such that wire feed channel 121 and passive guidewire feed surface 126 are vertically aligned with the upwardly open guidewire feed area 124 of housing 112, guidewire carriage 134 being movable between an open position 152 where feed tube 140 is spaced away from channel 118 on stanchion 117 (FIG. 22) of guidewire placement assembly 116 and a closed position 154 where feed tube 140 engages channel 118 on stanchion 117 (FIG. 21).

There is further provided (c) a sheathed guidewire 30 engaged with sheath brake 128 such that sheath 31 of the guidewire cannot move forwardly with respect to the sheath brake. The guidewire sheath is friction held in place by the sheath brake; although one may employ adhesive if so desired. Guidewire 20, which is freely movable within sheath 31, extends forwardly with respect to the sheath brake, over passive guidewire feed surface 126, through wire feed channel 121, channel 118 in stanchion 117 and hollow needle 22.

It is noted that guidewire 20 is untethered to device 110 and that device 110 does not include means for generating a vacuum. That is, there is no vacuum source communicating with hollow needle 22.

Current practice by many medical practitioners is to place a needle in a vessel/lumen while their other hand is controlling/maneuvering an ultrasound to locate the targeted area. Once the target is entered, the operator may observe fluid flashback through the needle hub and the subsequent drip of fluid (ascertaining visually the color and pressure of the fluid) onto the 'operating' surface, further confirming the location. To place the guidewire, the operator must subsequently discontinue holding the ultrasound and, at times, exchange with the hand holding the needle in order to engage the guidewire. At this juncture, the operator is unable to view, in real time, by themself, the guidewire entering the lumen of the vessel as well as the correct positioning of the guidewire. With the one-handed device of the invention, this inability to view the guidewire entering a lumen in real time is avoided.

The device of the invention may be packaged with a guidewire installed, in the open position, with the J-tip of the guidewire inside the wire feed channel 121. An operator may prefer to use the device in this open position and observe flashback through the transparent needle hub and subsequently observe fluid exiting the rear of the guidewire placement assembly 116 and dripping onto the operating surface. Once this flashback is observed, the operator can easily move the trigger to the closed position for subsequent guidewire placement.

Alternatively, the device may be packaged with a guidewire installed, in the closed position, with the J-tip of the guidewire inside the wire feed channel 121. An operator may prefer to use the device in closed position and observe flashback through a transparent needle hub as the operator can view concomitantly by ultrasound (or other imaging modalities), the current location of the tip of the needle. Moving the device from a closed position to an open position with the needle inserted into a patient is not recommended.

The device of the invention may also be used for non-fluid procedures -- such as targeting tumors and so forth as noted above.

Various features in any embodiment include wherein the passive guidewire feed surface 126 is on a freely rotating wheel 127, wherein the sheath brake projects upwardly from the upper surface 135 of guidewire carriage 134 having a channel 156 therethrough and wherein channel 156 through the sheath brake, the passive guidewire feed surface 126, wire feed channel 121, channel 118 through stanchion 117, microvalve assemblies 88A, 118A and hollow needle 22 are all substantially colinear. Further features include wherein channel 118 through stanchion 117 is conical in shape, and wherein wire feed channel 121 is a conical channel, and where hollow needle 22 has a transparent hub 22A.

A passive flashback device of FIGS. 19 through 34 may have a trigger 66 attached to guidewire carriage 134 as shown which is configured to move the guidewire carriage forwardly and rearwardly. The trigger may be of bifurcated construction with a forward member being forwardly concave and a rearward member being rearwardly concave so that the carriage may be easily triggered in either direction, or in other words in a "Y" shape, as shown.

FIG. 34 illustrates passive flashback device 110 in the closed position 154 grasped by a user, wherein it is seen that the device is operated with one hand, with digits of the user holding handle 114, a digit of the same hand operating trigger 66, and another digit of the same hand adjacent passive guidewire feed surface 126, which said other digit can be used to advance guidewire 20. It is appreciated from FIG. 34 and the discussion herein that housing 112, guidewire carriage 134, sheath brake 128, wire feed channel 121, and hollow needle 22 are all positioned, configured, and adapted for one-handed placement of a needle and guidewire in a patient.

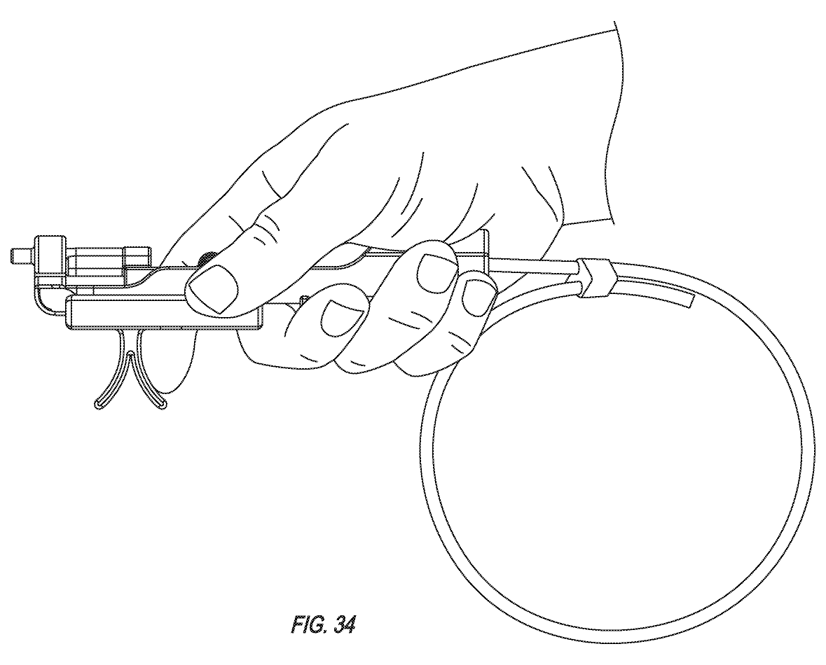

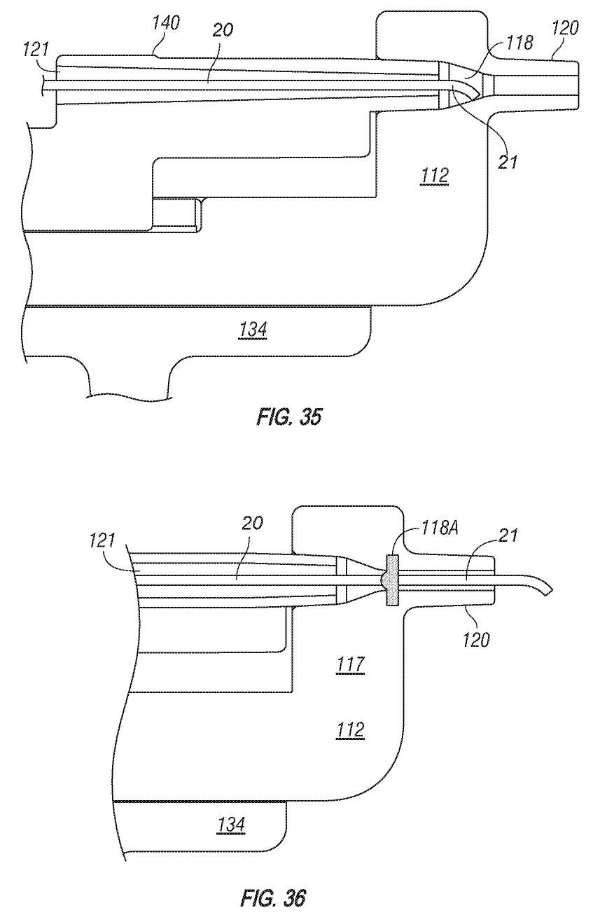

Guidewires typically have a J bend 21 at the end intended for insertion into a patient, so as not to inadvertently puncture a structure in a patient, such as a lumen of a blood vessel. The J bend can cause jamming of the guidewire in the device if the channels through which the unsheathed guidewire 20 is threaded are not properly configured, that is, the J bend should not be allowed to curl in the device to the extent which may cause blockage. In FIG. 35, it is seen when wire feed channel 121 and needle mounting projection channel 118 are properly sized, the J bend in wire 20 passes through an "eye" in the conical channel 118 of the mounting projection, in a substantially linear configuration and does not recover the J bend within the device. Preferred constructions of the invention use conical channels where appropriate, preferably sloping to a point in areas where the guidewire 20 is transferred from one conduit to another. It is noted that a J bend in the guidewire is bent to a 180° change in direction about its axis during fabrication and will return to this geometry when not restrained. The various guide channels in the device, including the wire feed channel, the channel through the stanchion, and the needle hub as well as the guidewire itself are positioned, configured and dimensioned so that the J bend is restrained from assuming its manufactured shape during use, that is prior to advancing the guidewire into the patient. Preferably the various guide channels are configured to allow for no more than 90° change in direction about the guidewire axis at the J bend as the guidewire passes through the device, more preferably, less than about 30° change in direction or less at the J bend as the wire passes through the device is believed suitable.

FIG. 36 is a schematic diagram illustrating a microvalve assembly 118A installed on the device of FIGS. 19, 35 about guidewire 20 proximate to hollow needle mounting projection 120. The microvalve assembly installation is the same as in the device of FIG. 17 (except for positioning as shown and described). The microvalve is located in channel 118, proximal/forward of a fully engaged feed tube 140 — as shown in the various drawings. Further details appear in FIG. 18 and the discussion relating thereto. The guidewire has a degree of interference with one or both seals of microvalve assembly 118A of anywhere from about 15% to 80%, with from about 35% to about 55% being more preferred. Besides the degree of interference of the guidewire with the elastic seal, it is also important not to distort the seals during assembly of the devices. To this end, the retaining surfaces do not compress the valves more than about 7.5–15% from their original dimensions, regardless of assembly method, whether threaded and screwed together or snap-fit with appropriate features on the device components. To this end, the retaining surfaces are of a predetermined axial distance with respect to one another of no less than about 85%–92.5% of the thickness of the peripheries of the valve assembly components prior to compression.

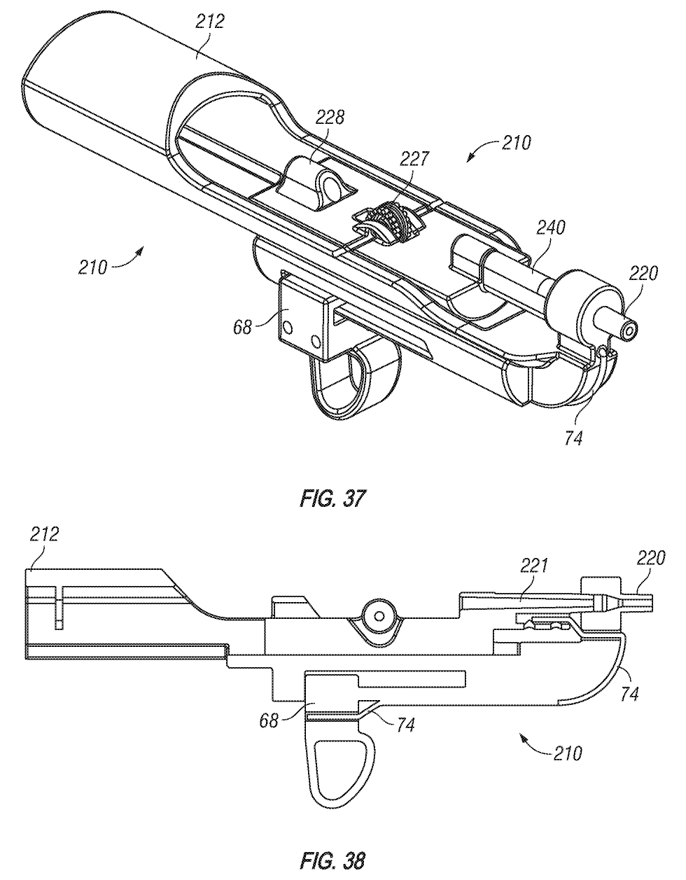

Alternatively, a trigger assembly 68 may be provided as shown in FIGS. 37 through 44, wherein corresponding parts of the device are numbered 100 numerals higher than in FIGS. 19–33. In the embodiment of FIGS. 37 through 44 assembly 68 is slidingly coupled to the guidewire carriage. Assembly 68 is configured to move the guidewire carriage forwardly and rearwardly. In particular, assembly 68 is mounted to slots 70, 72 on carriage 234, has an actuator wire 74 which bears against housing 212 as shown, and attached to the underside of the carriage and wire feed tube 240.

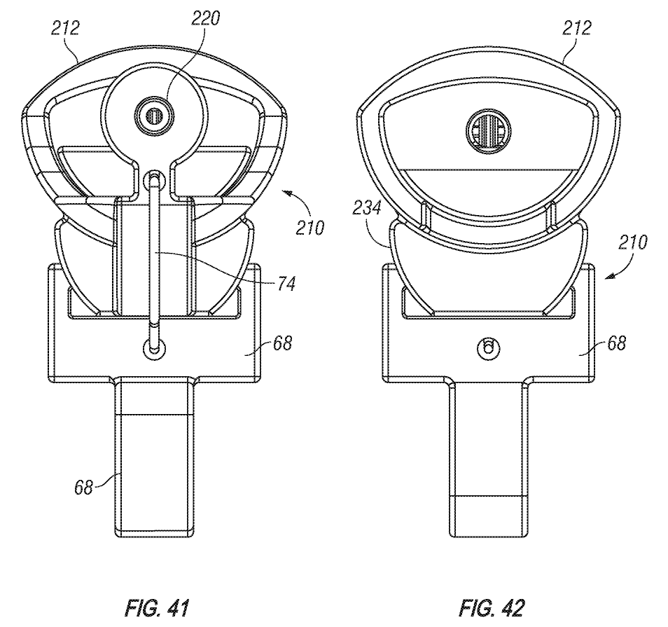

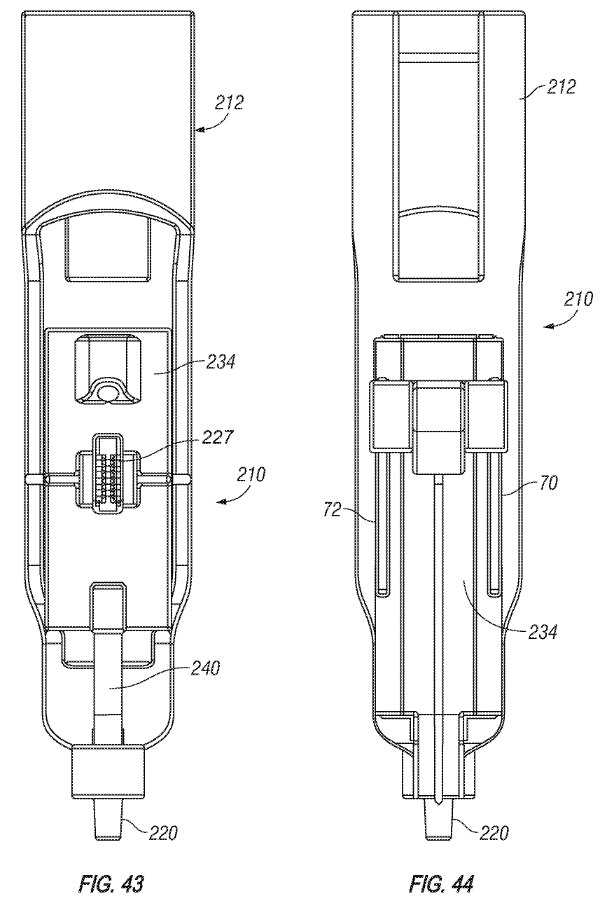

FIGS. 41 and 42 are front and rear views in elevation of device 210, respectively. FIGS. 43 and 44 are top and bottom plan views of device 210 in the closed position, respectively. These views further illustrate the relationship between elongate housing 212, guidewire carriage 234, trigger assembly 68, actuator wire 74, needle mounting projection 220, rotating wheel 227, sheath brake 228, and feed tube 240.

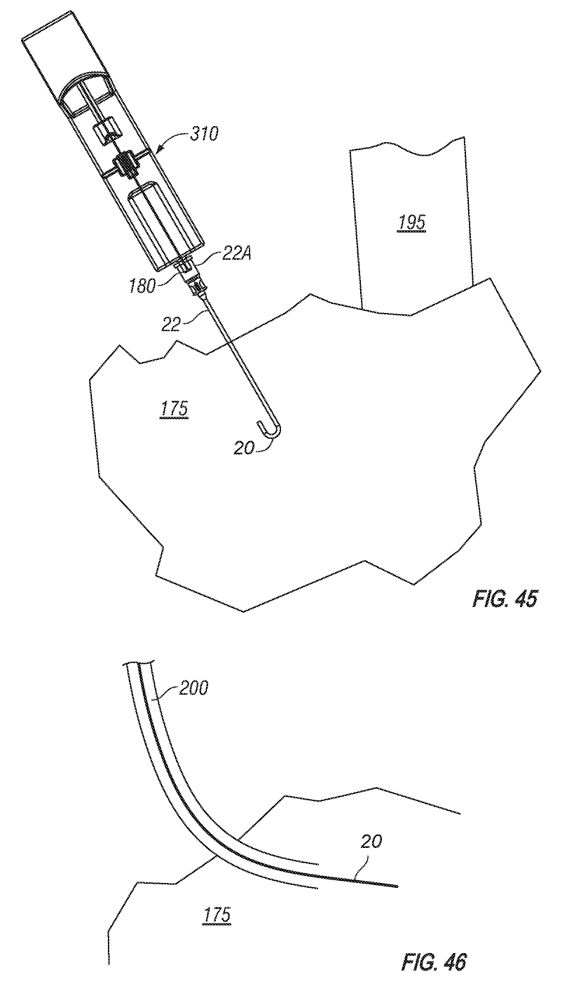

FIGS. 45 and 46 schematically illustrate a process for inserting a guidewire into a patient, whether device 10, 80, 110, 210, or 310 is used in combination with a needle and guidewire (see also FIGS. 1, 9, 19, and 37, for example). After needle 22 is properly placed, a central line 200 is inserted into the patient over the guidewire. For purposes of illustration, device 80 or device 110 in combination with a needle and guidewire are referred to as device 310, with features numbered as in the discussion above.

In particular, FIGS. 45 and 46 show a method of inserting a guidewire 20 into the body of a patient indicated at 175 comprising the steps of: (a) providing a passive flashback device 310 of the invention, said device being operated with one gloved hand having digits, (b) advancing a hollow bare needle indicated at 22 into patient 175 beneath the skin; (c) manipulating a probe or transducer 195 with the other hand to visualize the location of said hollow bare needle within patient 175 as the needle is advanced; (d) confirming proper placement by viewing the needle entering the lumen or vessel utilizing ultrasound; (e) in some cases, also confirming proper placement by observation of entry of a fluid 180, whether liquid, gaseous, or mixture, from the patient into a hub 22A (transparent or translucent) of the hollow needle 22; (f) advancing guidewire 20 threaded through said device into the patient by direct tactile contact between at least one digit of said gloved hand and said guidewire, while continuing to use said probe or transducer 195 to monitor the location of said guidewire within patient 175; and (g) stabilizing said guidewire in position within said patient without using said gloved hand operating said device; and (h) withdrawing said device. Preferably both hands are covered in sterile gloves.

Once the guidewire is inserted, a central line 200 may be placed over guidewire 20 and passed into said patient after the device has been withdrawn, as is shown in FIG. 34.

The invention further encompasses the following embodiments:

Embodiment 1. A device for introducing a guidewire into a patient configured for one-handed operation comprising:
(a) an elongate housing having
   (i) a handle at its rearward end,
   (ii) a guidewire placement assembly at its forward end with a wire feed channel communicating with a mounting projection for a hollow needle, said elongate housing further defining an upwardly open guidewire feed area;
(b) a guidewire passive feed surface disposed rearwardly of the guidewire placement assembly and vertically aligned with the upwardly open guidewire feed area;
(c) a guidewire sheath brake disposed rearwardly of the guidewire passive feed surface; and
(d) an elastomeric microvalve assembly disposed in the wire feed channel.

Embodiment 2. The device according to Embodiment 1, wherein the passive feed surface is on a rotatably mounted wheel.

Embodiment 3. The device according to Embodiment 1 or Embodiment 2, wherein the wire feed channel through the guidewire placement assembly is conical or tubular.

Embodiment 4. The device according to any of Embodiments 1, 2, or 3, in combination with a hollow needle affixed to the mounting projection of the guidewire placement assembly.

Embodiment 5. The combination according to Embodiment 4, in further combination with a sheathed guidewire inserted through the device, such that the sheath engages the sheath brake, and unsheathed guidewire extends over the guidewire passive feed surface and through the hollow needle, whereby the guidewire can be advanced into the patient with one hand.

Embodiment 6. The combination according to Embodiment 5, wherein the guidewire has a degree of interference with one of the seals of the microvalve assembly of anywhere from about 15% to 80%.

Embodiment 7. A passive flashback device for introducing a guidewire into a patient configured for one-handed operation comprising:
(a) an elongate housing having
   (i) a handle at its rearward end,
   (ii) a guidewire placement assembly at its forward end including a stanchion with a channel therethrough communicating with a hollow needle mounting projection which has a hollow needle mounted thereon communicating with the channel and
   (iii) said elongate housing further defining an upwardly open guidewire feed area;
(b) a guidewire carriage slidingly coupled to said elongate housing, the guidewire carriage having on its upper surface
   (i) a sheath brake,
   (ii) a wire feed channel and
   (iii) a passive guidewire feed surface, the guidewire carriage being positioned such that the wire feed channel and passive guidewire feed surface are vertically aligned with the upwardly open guidewire feed area of the housing, the guidewire carriage being movable between an open position where the wire feed channel is spaced away from the channel on the stanchion of the guidewire placement assembly and a closed position where the wire feed channel engages the channel on the stanchion;
(c) a sheathed guidewire engaged with the sheath brake such that the sheath of the guidewire cannot move forwardly with respect to the sheath brake, and the guidewire, which is freely movable within the sheath, extends forwardly with respect to the sheath brake, over the passive guidewire feed surface, through the wire feed channel, the channel in the stanchion and through the hollow needle; and
(d) an elastomeric microvalve assembly disposed in the channel in the stanchion,
wherein the guidewire is untethered to the device and wherein the device does not include means for generating a vacuum.

Embodiment 7A. The passive flashback device according to Embodiment 7, wherein the channel through the sheath brake, the passive guidewire feed surface, the wire feed channel, the channel through the stanchion, and the hollow needle are all substantially colinear.

Embodiment 8. The passive flashback device according to Embodiment 7, wherein the passive guidewire feed surface is on a freely rotating wheel.

Embodiment 9. The passive flashback device according to Embodiment 7 or Embodiment 8, wherein the sheath brake has a channel therethrough and the sheath brake projects upwardly from the upper surface of the guidewire carriage.

Embodiment 10. The passive flashback device according to any of Embodiments 7 through 9, wherein the wire feed channel is a conical channel.

Embodiment 11. The passive flashback device according to any of Embodiments 7 through 10, wherein the guidewire carriage has a trigger attached thereto configured to move the guidewire carriage forwardly and rearwardly.

Embodiment 12. The passive flashback device according to any of Embodiments 7 through 11, wherein the guidewire has a degree of interference with one of the seals of the microvalve assembly of anywhere from about 15% to 80%.

Embodiment 13. An assembly for receiving a sheathed guidewire in a passive flashback device for introducing a guidewire into a patient configured for one-handed operation comprising:
(a) an elongate housing having
   (i) a handle at its rearward end,
   (ii) a guidewire placement assembly at its forward end including a stanchion with a channel therethrough communicating with a hollow needle mounting projection which has a hollow needle mounted thereon communicating with the channel,
   (iii) said elongate housing further defining an upwardly open guidewire feed area, and (iv) an elastomeric microvalve assembly disposed in the channel through the stanchion; and
(b) a guidewire carriage slidingly coupled to said elongate housing, the guidewire carriage having on its upper surface
   (i) a sheath brake,
   (ii) a wire feed channel extending through a feed tube and
   (iii) a passive guidewire feed surface, the guidewire carriage being positioned such that the wire feed channel and passive guidewire feed surface are vertically aligned with the upwardly open guidewire feed area of the housing, the guidewire carriage being movable between an open position where the feed tube is spaced away from the channel on the stanchion of the guidewire placement assembly and a closed position where the feed tube engages the channel on the stanchion of the guidewire placement assembly.

Embodiment 14. A device for one-handed insertion of a guidewire comprising:
(a) a mounting/gripping member having disposed thereon a syringe with a barrel and a plunger with a guidewire passage therethrough and a valve assembly located within the plunger;
(b) an actuator carriage slidably engaged to the mounting/gripping member having a guidewire feed and a trigger, the actuator carriage also being engaged to the plunger of the syringe so as to axially move the plunger of the syringe forwardly and backwardly by manipulation of the trigger and sliding motion of the actuator carriage with respect to the mounting/gripping member; and
(c) a guidewire installed through the plunger,
wherein the guidewire engages the valve assembly which has at least one elastic seal in an interference fit with the guidewire and wherein said guidewire feed and said trigger are disposed relative to said mounting/gripping member such that when the mounting/gripping member is engaged by one hand, a digit of said one hand is able to urge the trigger of said actuator carriage rearwardly and/or forwardly, and between the digit of said one hand and the guidewire feed, another digit of said one hand is able to engage the guidewire to advance the guidewire through said syringe, sensing the degree of resistance encountered by a portion of said guidewire.

Embodiment 15. The device according to Embodiment 14, wherein the at least one elastic seal in an interference fit with the guidewire has a degree of interference with the guidewire from about 15% to about 80%.

Embodiment 16. A syringe with a guidewire, comprising:
(a) a syringe with a barrel and a plunger with a guidewire passage therethrough and a valve assembly located within the plunger; and
(b) a guidewire installed through the plunger,
wherein the guidewire engages the valve assembly which has at least one elastic seal in an interference fit with the guidewire.

Embodiment 17. In a medical procedure for the diagnosis and/or treatment of a medical condition comprising inserting a hollow needle into a patient, observing the needle enter the lumen or vessel utilizing an imaging modality such as ultrasound while the needle is being advanced to confirm placement of the needle, optionally observing fluid or blood flashback to a hub of the needle, and subsequently advancing a guidewire through the needle into the patient while continuing to image the position of the guidewire in the patient with the imaging modality, the improvement comprising utilizing the passive flashback device of any of Embodiments 7–12 or the combination of Embodiment 5 to insert the guidewire.

Embodiment 18. The improvement according to Embodiment 17, wherein the imaging modality is selected from the group consisting of ultrasound, fluoroscopy, X-ray, and computed tomography (CT) scanning.

Embodiment 19. The improvement according to Embodiment 17 or Embodiment 18, wherein the medical procedure is selected from the group consisting of central line placement, nephrostomy placement, uterine fibroid embolization, tumor ablation, embolization of the blood supply to a tumor, endovenous ablation for varicose veins, deep vein thrombosis treatment, IVC filter removal, peripheral arterial disease treatment, retrograde intubation, chest tube placement, and blood draw, as well as any other Seldinger technique.

While the invention has been described in detail, modifications within the spirit and scope of the invention will be readily apparent to those of skill in the art. Such modifications are also to be considered as part of the present invention. In view of the foregoing discussion, relevant knowledge in the art and references discussed above in connection with the Background of the Invention and Detailed Description, the disclosures of which are all incorporated herein by reference, further description is deemed unnecessary. In addition, it should be understood that aspects of the invention and portions of various embodiments may be combined or interchanged either in whole or in part. Furthermore, those of ordinary skill in the art will appreciate that the foregoing description is by way of illustration only and is not intended to limit the invention.

What is claimed is:

1. A device for introducing a guidewire into a patient configured for one-handed operation comprising:
(a) an elongate housing having (i) a handle at its rearward end, (ii) a guidewire placement assembly at its forward end with a wire feed channel communicating with a mounting projection for a hollow needle, said elongate housing further defining an upwardly open guidewire feed area;
(b) a guidewire passive feed surface disposed rearwardly of the guidewire placement assembly and vertically aligned with the upwardly open guidewire feed area; and
(c) a guidewire sheath brake disposed rearwardly of the guidewire passive feed surface.

2. The device according to claim 1, wherein the guidewire passive feed surface is on a rotatably mounted wheel.

3. The device according to claim 1, wherein the wire feed channel through the guidewire placement assembly is conical or tubular.

4. The device according to claim 1, further comprising an elastomeric microvalve assembly disposed in the wire feed channel.

5. The device according to claim 1, in combination with a hollow needle affixed to the mounting projection of the guidewire placement assembly.

6. The combination according to claim 5, in further combination with a sheathed guidewire inserted through the device, such that a sheath of the sheathed guidewire engages the guidewire sheath brake, and unsheathed guidewire extends over the guidewire passive feed surface and through the hollow needle, whereby the guidewire can be advanced into the patient with one hand.

7. A passive flashback device for introducing a guidewire into a patient configured for one-handed operation comprising:
(a) an elongate housing having (i) a handle at its rearward end, (ii) a guidewire placement assembly at its forward end including a stanchion with a channel therethrough communicating with a hollow needle mounting projection which has a hollow needle mounted thereon communicating with the channel and (iii) said elongate housing further defining an upwardly open guidewire feed area;
(b) a guidewire carriage slidingly coupled to said elongate housing, the guidewire carriage having on its upper surface (i) a sheath brake, (ii) a wire feed channel and (iii) a passive guidewire feed surface, the guidewire carriage being positioned such that the wire feed channel and passive guidewire feed surface are vertically aligned with the upwardly open guidewire feed area of the elongate housing, the guidewire carriage being movable between an open position where the wire feed channel is spaced away from the channel on the stanchion of the guidewire placement assembly and a closed position where the wire feed channel engages the channel on the stanchion; and
(c) a sheathed guidewire engaged with the sheath brake such that a sheath of the sheathed guidewire cannot move forwardly with respect to the sheath brake, and the guidewire, which is freely movable within the sheath, extends forwardly with respect to the sheath brake, over the passive guidewire feed surface, through the wire feed channel, the channel in the stanchion and through the hollow needle;
wherein the guidewire is untethered to the passive flashback device and wherein the passive flashback device does not include means for generating a vacuum.

8. The passive flashback device according to claim 7, wherein the channel through the sheath brake, the passive guidewire feed surface, the wire feed channel, the channel through the stanchion, and the hollow needle are all substantially colinear.

9. The passive flashback device according to claim 7, wherein the passive guidewire feed surface is on a freely rotating wheel.

10. The passive flashback device according to claim 7, wherein the sheath brake has a channel therethrough and the sheath brake projects upwardly from the upper surface of the guidewire carriage.

11. The passive flashback device according to claim 7, wherein the wire feed channel is a conical channel.

12. The passive flashback device according to claim 7, wherein the guidewire carriage has a trigger attached thereto configured to move the guidewire carriage forwardly and rearwardly.

13. The passive flashback device according to claim 12, wherein the trigger is Y-shaped.

14. The passive flashback device according to claim 7, further comprising an elastomeric microvalve assembly disposed in the channel through the stanchion, wherein the guidewire has a degree of interference with a seal of the elastomeric microvalve assembly of anywhere from about 15% to 80%.

15. The passive flashback device according to claim 7, further comprising a trigger assembly slidingly coupled to the guidewire carriage via slots on the guidewire carriage, the trigger assembly having an actuator wire configured to bear against the elongate housing to move the guidewire carriage forwardly and rearwardly.

16. An assembly for receiving a sheathed guidewire in a passive flashback device for introducing a guidewire into a patient configured for one-handed operation comprising:
(a) an elongate housing having (i) a handle at its rearward end, (ii) a guidewire placement assembly at its forward end including a stanchion with a channel therethrough communicating with a hollow needle mounting projection which has a hollow needle mounted thereon communicating with the channel, and (iii) said elongate housing further defining an upwardly open guidewire feed area; and
(b) a guidewire carriage slidingly coupled to said elongate housing, the guidewire carriage having on its upper surface (i) a sheath brake, (ii) a wire feed channel and (iii) a passive guidewire feed surface, the guidewire carriage being positioned such that the wire feed channel and passive guidewire feed surface are vertically aligned with the upwardly open guidewire feed area of the elongate housing, the guidewire carriage being movable between an open position where the wire feed channel is spaced away from the channel on the stanchion of the guidewire placement assembly and a closed position where the wire feed channel engages the channel on the stanchion of the guidewire placement assembly.

17. The assembly according to claim 16, further comprising an elastomeric microvalve assembly disposed in the channel through the stanchion.

18. The assembly according to claim 16, wherein the guidewire carriage has a trigger attached thereto or a trigger assembly slidingly coupled thereto, configured to move the guidewire carriage forwardly and rearwardly.

19. In a medical procedure for diagnosis and/or treatment of a medical condition comprising inserting a hollow needle into a patient, observing the needle enter the lumen or vessel utilizing an imaging modality while the needle is being advanced to confirm placement of the needle, optionally observing fluid or blood flashback to a hub of the hollow needle, and subsequently advancing a guidewire through the hollow needle into the patient while continuing to image the position of the guidewire in the patient with the imaging modality, the improvement comprising utilizing the device of claim 1 to insert the guidewire, wherein the hollow needle is affixed to the mounting projection of the guidewire placement assembly.

20. The improvement according to claim 19, wherein the imaging modality is selected from the group consisting of ultrasound, fluoroscopy, X-ray, and computed tomography (CT) scanning.

21. The improvement according to claim 19, wherein the step of observing fluid or blood flashback to the hub occurs after confirming placement of the needle utilizing the imaging modality and before advancing the guidewire through the hollow needle into the patient.
